(19) United States
(12) Patent Application Publication
(10) Pub. No.: US 2025/0189475 A1
(43) Pub. Date: Jun. 12, 2025

(54) CAPACITIVE SENSING FILM, RELATED INTELLIGENT PAPER DIAPER, AND DETECTION SYSTEM DEVICE

(71) Applicants: Shenzhen Ediaper Technology Limited, Shenzhen, Guanagdong (CN); Sanwa Corporation, Tokyo (JP)

(72) Inventors: Sun Hoi WONG, Hong Kong (CN); Fei XU, Hong Kong (CN); Zhen CHEN, Hong Kong (CN)

(21) Appl. No.: 18/844,943

(22) PCT Filed: May 9, 2023
(86) PCT No.: PCT/CN2023/093022

§ 371 Date: Sep. 6, 2024

(30) Foreign Application Priority Data
Mar. 9, 2022 (CN) 202210226345.3

(51) Int. Cl. G01N 27/22; A61F 13/42

(52) U.S. Cl. CPC G01N 27/227; A61F 13/42; G01N 27/228

(57) ABSTRACT

A capacitive sensing film, a smart diaper and a wetness detection system device including the capacitive sensing film. The capacitive sensing film includes a metal film electrode, a flexible substrate, and a waterproof protective layer; the metal film electrode is deposited on the flexible substrate, and the waterproof protective layer covers the metal film electrode and forms an interlayer, adapted to protect the metal film electrode; the metal film electrode has a smooth surface and a nanoscale thickness, adapted to reduce the surface area in contact with a liquid through the gap of the interlayer; when the capacitive sensing film is wetted by the liquid containing electrolyte, the electric double layer capacitance generated between the metal film electrode and the liquid is smaller than the electrolytic capacitance generated between the metal film electrode and the liquid, thus a wetness detection function based on electrolytic capacitance is realized.

### BACKGROUND

### Priority Claim

This application claims the priority of the patent application No. 202210226345.3 for the invention entitled “Capacitive sensing film, related smart diaper and detection system device” filed with the Chinese Patent Office on Mar. 9, 2022, all of which are combined by reference in this application.

### TECHNICAL FIELD

The present invention relates to a sensing film, in particular to a capacitive sensing film with anti-short-circuit, anti-damage and strong anti-interference capabilities, a smart diaper and a wetness detection system device related to the capacitive sensing film.

### RELATED ART

Disposable absorbent articles include diapers, insertion pads, pull-up pants, toddler pants, changing pads, sanitary napkins, maternity napkins, etc. Because it is a disposable hygiene product, its use needs to face the problem of timely replacement, if the replacement is too frequent, it is not only troublesome and wasteful; and if the replacement is too late, it is easy to cause leakage and skin problems such as diaper rash. To solve these problems, there is a need for a sensor to detect the wetness state of disposable absorbent articles and to prompt for replacement, which has positive significance for the scientific use of disposable absorbent articles.

In the prior art, Chinese Patent Application Publication No. CN111077192A discloses an excrement sensor and a manufacturing method, wherein 2-3 detection electrodes printed with carbon paddles are disposed in parallel in an interlayer composed of upper and lower waterproof films, thereby forming a capacitive film sensor for detecting excrement, especially for the detection of wetness caused by urine, loose stool, etc. Since it is a soft film, it can be called a sensing strip or sensing film.

Since the sensing film is soft and thin, it can be rolled up into roll form and used as a production material, which is then cut and placed into the disposable absorbent article during the production process of the disposable absorbent article. However, this sensing film has a disadvantage that when the excrement wets the cross-section, there will be a large electric double layer capacitance interference between the two detection electrodes, or it will cause the electrodes to short circuit and cause false alarms. In addition, the sensing film is a easily damaged film, which is prone to rupture during production and use. When the damaged part is wetted by excrement, it will also cause great interference or false alarms.

In addition, the above-mentioned prior art is also relatively susceptible to electromagnetic interference, especially power frequency interference, because it has longer electrodes and higher internal resistance, and when in use, it is close to human skin and soaked in excrement, these factors lead to more serious interference, which makes the detection data unstable and reduces the reliability of wetness detection. The market demand and the deficiencies of traditional technology need to be solved by new technical solutions.

### SUMMARY

The technical problem to be solved by the present invention is to provide a capacitive sensing film that can effectively resist short circuit, damage, and strong interference, when it is cut off and put into a disposable absorbent article for use, no matter whether there is excrement that wets the cross section and longitudinal section, no matter whether its sensing area is damaged, or whether it is strongly disturbed by the human body or the environment, it should be able to perform wetness detection work normally, stably and reliably.

In order to solve the above technical problems, in a first aspect, the present invention provides a capacitive sensing film (hereinafter referred to as sensing film), including a metal film electrode, a flexible substrate and a waterproof protective layer, the metal film electrode is deposited on the flexible substrate, and the waterproof protective layer covers the metal film electrode and forms a waterproof interlayer together with the flexible substrate, adapted to protect the metal film electrode, the metal film electrode has a smooth surface and a nanoscale thickness, adapted to reduce the surface area of the metal film electrode in contact with the liquid through the gap of the interlayer at the edge or damaged part of the sensing film, thereby inhibiting the generation of electric double layer capacitance at liquid-solid contact interface; when the sensing film is wetted by a liquid containing electrolyte, the electric double layer capacitance generated between the metal film electrode and the liquid is smaller or much smaller than the electrolytic capacitance generated between the metal film electrode and the liquid, therefore, the interference or influence of the electric double layer capacitance on the normal wetness detection of the sensing film is reduced or eliminated.

The metal film electrode may include a metal film electrode formed by physical vapor deposition, and is configured to detect the degree that the sensing film is wetted by the liquid by means of electrolytic capacitance, the metal film electrode is preferably a strip shape and a very small thickness-to-width ratio, so as to increase its ability to generate electrolytic capacitance based on width and suppress its ability to generate electric double layer capacitance based on thickness, therefore the sensing film has an ability to detect the degree of wetness by means of electrolytic capacitance.
the sensing film may include at least one cross section or longitudinal section, and the metal film electrode is exposed through the gap of the interlayer on the cross section or longitudinal section, when the sensing film is completely wetted by the liquid, the electrolytic capacitance generated between the metal film electrode and the liquid is bigger or much bigger than the electric double layer capacitance generated between the metal film electrode and the liquid on the gap of the interlayer of the cross section or longitudinal section; or

When the sensing film is cut along the center line of the metal film electrode to obtain left and right incised sensing films and corresponding left and right metal film electrodes, the left and right metal film electrodes can be exposed through the gap of the interlayer on the incision, and when the left and right incised sensing films are fully wetted by the liquid, the electrolytic capacitance generated between the left and right metal film electrodes is bigger or much bigger than the electric double layer capacitance generated between the left and right metal film electrodes.

The metal film electrode is preferably a vacuum evaporated metal film electrode, and preferable a thickness-to-width ratio of 1/10,000 to 1/100,000 or less, adapted to suppress the electric double layer capacitance generated at the edge, cut or damaged part of the sensing film, so that the sensing film has the ability to resist short circuit and damage; and the metal film electrode preferably has a square resistance of 0.1 ohm to 50 ohm or less, adapted to suppress the electromagnetic interference generated inside the metal film electrode, so that the sensing film has strong anti-interference ability.

The thickness of the waterproof protective layer is preferably smaller or much smaller than the thickness of the flexible substrate, adapted to improve the ability of the sensing film to generate electrolytic capacitance in the direction of the waterproof protective layer; when the sensing film is fully wetted by the liquid, the electrolytic capacitance generated by the liquid on the outer surface of the waterproof protective layer is bigger or much bigger than the electrolytic capacitance generated by the liquid on the outer surface of the flexible substrate, thereby realizing the wetness detection function focusing on the direction of the waterproof protective layer.

The sensing film can be selected in the shape of a strip and includes at least two of the metal film electrodes and constitutes a first detection electrode and a second detection electrode, wherein at least one of the detection electrodes is located in the interlayer formed by the flexible substrate and the waterproof protective layer; when the sensing film is wetted by a liquid containing electrolyte, the electrolytic capacitance generated between the first and second detection electrodes is bigger or much bigger than the electric double layer capacitance generated between the first and second detection electrodes, the sensing film is configured to realize the function of detecting the degree of wetness through the detection of electrolytic capacitance between the first and second detection electrodes, the degree of wetness is proportional to the electrolytic capacitance between the first and second detection electrodes.

The width of the sensing film is preferably in the range of 5 to 100 mm; the width of the first and second detection electrodes is preferably in the range of 1 to 50 mm; the length of the sensing film is preferably in the range of 5 to 120 cm; one end of the sensing film may include a bare area, where the first and second detection electrodes are exposed, adapted to be electrically connected with a detection device and realize the wetness detection function.

The sensing film may also include a waterproof covering layer, which may include a non-woven fabric, a plastic film or a waterproof paper, the waterproof covering layer covers the bare area, and together with the bare area constitutes a pocket for accommodating the detection device.

The sensing film may also include a RFID tag, the RFID tag may include a RFID chip and a RFID antenna, and the RFID chip is configured to be electrically connected to the first and second detection electrodes, and the information related to the wetness is obtained by detecting the electrolytic capacitance between the first and second detection electrodes, and the RFID antenna is adapted to send the information to the outside by means of radio frequency.

The flexible substrate is preferably a plastic film; the metal film electrode is further preferably a vacuum evaporated aluminum film electrode; the thickness of the metal film electrode is preferably in the range of 5 to 250 nanometers; the thickness of the waterproof protective layer is preferably 0.25 to 5 microns; the thickness of the flexible substrate is preferably 5 to 250 microns; the liquid containing electrolyte may include urine, loose stool, sweat, menstrual blood or saline; the much bigger is preferably in the range of 3 to 30 times or more; the much smaller is preferably in the range of ⅓ to 1/30 or less; the waterproof protective layer may include polymer coating layer, waterproof printing layer, plastic composite layer or extrusion laminating layer.

In another aspect, the present invention provides a smart diaper, which in addition to the above-mentioned sensing film, also includes a disposable absorbent article, which may include a top layer, an absorbent layer and a leak-proof layer, and the sensing film may be disposed in the disposable absorbent article and is configured to realize quantitative wetness detection function by means of electrolytic capacitance detection.

The sensing film may include a color positioning mark, adapted to guide the production equipment to cut the sensing film and the disposable absorbent article together at a specific place, wherein the sensing film is cut into two sections, one long section and one short section, the long section is the main part, and the short end is the residual part, the main part may include a sensing area and a bare area, the sensing area may be located at the crotch portion of the disposable absorbent article, and the beginning of the bare area is flush with the front edge of the disposable absorbent article.

The disposable absorbent article may include diapers, insertion pads, pull-up pants, training pants, sanitary napkins, maternity napkins, or urine pads; the absorption layer may include a polymer absorption material, the top layer may include a hydrophilic non-woven fabric, and the leak-proof layer may include a breathable or non-breathable polyethylene film.

In still another aspect, the present invention provides a wetness detection system device, which in addition to the above-mentioned sensing film, may also include a detection device and a disposable absorbent article, the disposable absorbent article may include a surface layer, an absorbent layer, and a leak-proof layer, and the sensing film may be disposed in the disposable absorbent article, and the detection device is electrically connected with the first and second detection electrodes of the sensing film and is configured to realize quantitative wetness detection function of the disposable absorbent article by means of electrolytic capacitance.

The detection device may include a wireless transmitting device and a wireless receiving device for transmitting and receiving the wetness information of the disposable absorbent article and is adapted to realize wetness indication or wetness alarm function of the disposable absorbent article.

The wireless receiving device may include a dedicated sound or light alarm device, a smartphone, a PC, or a tablet.

The beneficial effect of the present invention is that, by replacing the traditional carbon electrode with a smooth and nanoscale metal film electrode, the thickness of the detection electrode and the surface area in contact with the liquid are greatly reduced, it effectively suppresses the generation of electric double layer capacitance on the cross section of the sensing film or at the damaged part. Even if the liquid containing electrolyte soaks the detection electrodes in these parts, it will not cause large electric double layer capacitance interference and will not cause short circuits between electrodes and false alarms.

At the same time, the present invention also adopts the method of greatly reducing the thickness to width ratio of the detection electrodes to increase the ratio of the electrolytic capacitance between the electrodes to the electric double layer capacitance, and successfully replaced the electric double layer capacitance with electrolytic capacitance to achieve more stable and reliable wetness detection, and its metal film electrode also greatly reduces the internal resistance of the detection electrode, which greatly improves the anti-interference ability of the sensing film. That is to say, the sensing film of the present invention has good anti-short-circuit, anti-damage, and anti-interference capabilities, and is able to provide a more stable and reliable solution for wetness detection and intelligent upgrade of disposable absorbent articles.

### BRIEF DESCRIPTION OF THE DRAWINGS

To explain the technical solutions of the embodiments of the present invention more clearly, the following briefly introduces the accompanying drawings required for the embodiments. Obviously, the drawings in the following description are only some embodiments of the present invention, for those of ordinary skill in the art, other drawings can also be obtained from these drawings without creative effort.

FIG. 1 is a schematic structural diagram of an intelligent (or smart) wetness detection system device (also be called system apparatus or simply system) according to an embodiment of the present invention (top view).

FIG. 2 is a schematic diagram (a perspective view) of a layered structure of a smart diaper according to an embodiment of the present invention.

FIG. 3 is a schematic diagram (a perspective view) of the appearance structure of a capacitive sensing film according to an embodiment of the present invention.

FIG. 4 is a schematic structural diagram (left view) of a capacitive sensing film according to an embodiment of the present invention.

FIG. 5 is a schematic structural diagram (front view) of a capacitive sensing film according to an embodiment of the present invention.

FIG. 6 is a schematic diagram of the A-A′ cross sectional structure of the capacitive sensing film according to the embodiment of the present invention shown in FIG. 5 and an equivalent circuit diagram of the same when it is fully wetted by a liquid containing electrolyte.

FIG. 7 is a schematic structural diagram (top view) of a cross section of a capacitive sensing film when it is wetted by a liquid containing electrolyte according to an embodiment of the present invention.

FIG. 8 is a schematic structural diagram (a perspective view) of one end of a capacitive sensing film including a bare area according to an embodiment of the present invention.

FIG. 9 is a schematic structural diagram (top view) of a capacitive sensing film including a positioning mark and disposed in a disposable absorbent article according to an embodiment of the present invention.

FIG. 10 is a schematic structural diagram (top view) when the main body of the detection electrode of a capacitive sensing film is completely covered and sealed according to an embodiment of the present invention.

FIG. 11 is a schematic structural diagram (top view) of a capacitive sensing film including a RFID tag according to an embodiment of the present invention.

FIG. 12 is a schematic structure diagram of a capacitive sensing film including only one metal film electrode according to an embodiment of the present invention, and an equivalent circuit diagram when it is fully wetted by a liquid containing electrolyte (cross sectional view).

FIG. 13 is a schematic structural diagram of a capacitive sensing film including a bare detection electrode and an equivalent circuit diagram (cross sectional view) when it is fully wetted by a liquid containing electrolyte according to an embodiment of the present invention.

FIG. 14 is a functional block diagram of an intelligent/smart wetness detection system device according to an embodiment of the present invention.

### DETAILED DESCRIPTION

The following descriptions of the various embodiments refer to the accompanying drawings to illustrate specific embodiments in which the invention may be practiced. The directional and positional terms mentioned in the present invention, such as “up”, “down”, “front”, “rear”, “left”, “right”, “inside”, “outside”, “top”, “bottom”, “side”, “front”, “head”, “tail”, “both ends”, “both sides”, etc., only refer to the direction or position of the drawings. Therefore, the directional and positional terms used are for describing and understanding the present invention, rather than limiting the protection scope of the present invention.

The present invention will be further described below in conjunction with the accompanying drawings. Referring to FIG. 1, this is a schematic structural diagram of an intelligent wetness detection system according to an embodiment of the present invention (top view), which includes a disposable absorbent article 10, it is a kind of disposable absorbent hygiene article including diapers, insertion pads, toddler pants, pull-up pants, changing pads, sanitary napkins, maternity napkins etc. Disposable absorbent articles typically include a top layer (inner layer, top sheet, dry layer, facing the user's skin when in use), leak-proof layer (anti-leakage layer, outer layer, back sheet, facing away from the user's skin when used) and absorbent layer (interlayer, moisture-absorbing layer, located between the top layer and the leak-proof layer) and other components.

In addition to the disposable absorbent article 10, this embodiment also includes a capacitive sensing film (referred to as sensing film, sensor film, inductive film, or induction film) 20 disposed in the disposable absorbent article. It is an excrement sensor/wetness sensor in the form of a film, usually in the shape of a long ribbon/strip, and usually includes two detection electrodes 21 and 22 parallel to each other, which can be referred to as a first detection electrode and a second detection electrode respectively. This embodiment also includes a wetness detection device (referred to as a detection device) 30, which is integrated with the first and second detection electrodes through electrical connections 34 for use. The detection device 30 includes a capacitance detection unit, which can realize the function of detecting the wetness of the absorbent article 10 by means of electrolytic capacitance detection.

In practical applications, both the absorbent article 10 and the sensing film 20 are disposable, while the detection device 30 is reusable. Since the disposable absorbent article including the sensing film 20 can realize intelligent wetness detection, it can be called an intelligent disposable absorbent article, and can also be collectively referred to as a smart diaper. When the detection device is matched with the smart diaper, an intelligent wetness detection system can be formed.

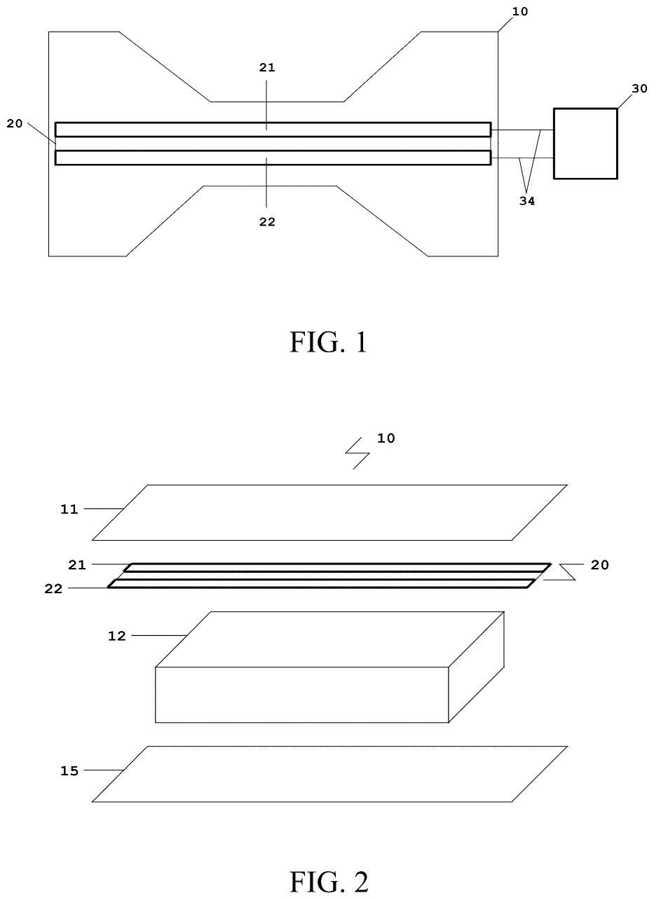

Referring to FIG. 2, this is a schematic diagram (a perspective view) of a layered structure of a smart diaper according to an embodiment of the present invention. The disposable absorbent article 10 of this embodiment includes a top layer 11, an absorbent layer 12 and a leak-proof layer 15. During use, the top layer 11 will be in contact with human skin, when the user urinates, the urine will enter the absorbent layer 12 through the hydrophilic and loose and breathable top layer (such as hydrophilic non-woven fabric), it is then absorbed by wood pulp and/or polymer absorbing materials (such as SAP) in the absorbing layer, wherein the SAP has the function of locking moisture, which can gradually restore the top layer 11 to dryness. As for the leak-proof layer 15, it is mainly used to prevent leakage of urine, and is usually made of waterproof breathable or non-breathable polyethylene film (PE film).

This embodiment also includes a sensing film 20, which includes a first detection electrode 21 and a second detection electrode 22, the sensing film 20 is disposed between the top layer 11 and the absorption layer 12 of the disposable absorbent article. In practical applications, the sensing film can also be disposed on the top layer of the disposable absorbent article, between the absorption layer and the leak-proof layer, or within the absorbing layer. The components of the smart diaper in this embodiment are drawn in a layered manner, in practical applications, the above components will be bonded together by adhesives (such as hot melt adhesives, structural adhesives), the sensing film 20 together with the disposable absorbent article (including top layer 11, absorbing layer 12 and leak-proof layer 15) constitute the smart diaper of the embodiment of the present invention. The length of the sensing film can be the same as that of the disposable absorbent article, or it can be longer or shorter, and it is usually in the range of 5 to 120 cm.

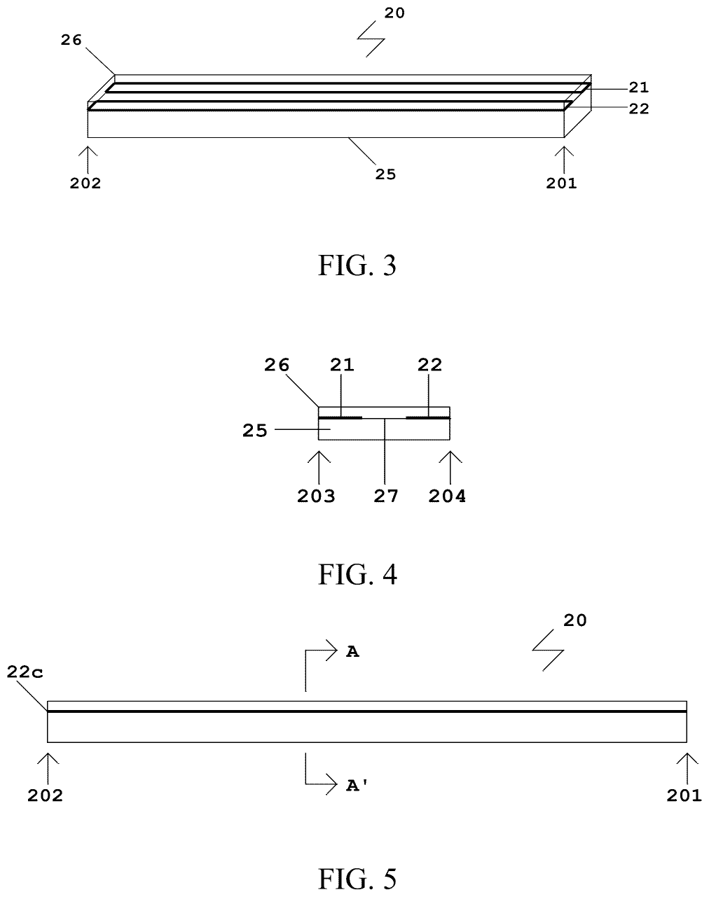

Referring to FIG. 3, which is a schematic diagram (a perspective view) of the appearance structure of a capacitive sensing film according to an embodiment of the present invention. Different from the prior art, which uses carbon paddles to print the detection electrodes on the waterproof film, and then covers the electrodes with another waterproof film, the first and second detection electrodes in the embodiment of the present invention are ultra-thin metal film electrodes, it can be produced by physical vapor deposition (Physical Vapor Deposition, PVD) on a flexible substrate, commonly used PVD methods include vacuum evaporation, radio frequency sputtering (RF sputtering), etc.

Through above methods, an ultra-thin layer of metal (such as gold, silver, copper, zinc, chromium, aluminum, etc., preferably pure aluminum) can be deposited on the flexible substrate and constitute the metal film electrode/detection electrode of the embodiment of the present invention, its thickness is usually only 5 to 250 nanometers (nanoscale), which is much thinner than traditional carbon paddle printing thickness of 1 to 25 micrometers (micron scale). Considering the cost-effectiveness, the embodiment of the present invention is preferably the vacuum evaporation method, especially an aluminum film electrode/aluminized film electrode generated by vacuum evaporation.

The flexible substrate 25 can be selected from plastic films such as PE (polyethylene), PP (polypropylene), CPP (cast polypropylene), BOPP (biaxially oriented polypropylene), and PET (polyester). In addition to being soft and comfortable, the flexible substrate also needs to have a certain strength and good waterproof performance, so the flexible substrate of the embodiment of the present invention can also be called a waterproof film. In order to obtain the desired detection electrode pattern, local evaporation technology may also be used in practical applications. For example, a blocking method is used to create a suitable gap between the metal film electrodes to form the first and second detection electrodes that are parallel to each other and are separated from each other or use a removal method to clean/remove the metal between the metal film electrodes to obtain the desired metal film electrode pattern. After the required detection electrodes are disposed on the flexible substrate, the waterproof protective layer 26 can be then coated on the detection electrodes, in this way, the flexible substrate 25 can form a waterproof interlayer together with the waterproof protective layer 26, and the first and second detection electrodes 21 and 22 can be covered up and down and protected.

In practical applications, the thickness of the flexible substrate is usually between 5 and 250 microns, while the thickness of the waterproof protective layer is between 0.25 and 5 microns. The thickness of the waterproof protective layer is smaller or much smaller than that of the flexible substrate. The sensing film 20 is generally designed in the shape of a long strip, and its beginning (cross section 201) to the ending end (cross section 202) represent the length of the sensing film. The original sensing film may also be a very long roll (for example, thousands of meters of rolled material), and the sensing film roll is cut and placed into the disposable absorbent article for use as needed during the production of the disposable absorbent article.

The waterproof protective layer in the embodiment of the present invention is a concept with a broad meaning. As long as a layer of waterproof material is covered on the first and second detection electrodes, it can be regarded as a kind of waterproof protective layer, including but not limited to polymer waterproof coating, waterproof printing, or generated by the composite process or lamination process of plastic film (such as PE) etc.

Referring to FIG. 4, which is a schematic structural diagram (left view) of a capacitive sensing film according to an embodiment of the present invention, it includes a flexible substrate 25, a waterproof protective layer 26, a first detection electrode 21, a second detection electrode 22, and an interlayer 27 formed by the flexible substrate and the waterproof protective layer. The primitive sensing film is usually a large roll of wide format sensing film, which will be assembled by many groups of sensing films, and then many rolls of narrow format sensing film are obtained by longitudinal slitting. Finally, the narrow format sensing film is cut transversely on the production line of disposable absorbent articles to obtain a sensing film with a length similar to that of disposable absorbent articles. That is to say, the sensing film of the embodiment of the present invention is usually produced by “cutting”, and its longitudinal sections 203 and 204 are parallel to the first and second detection electrodes, the first and second detection electrodes 21 and 22 are exposed through the gap of the interlayer in the longitudinal sections. As for the cross sections 201 and 202 are perpendicular to the first and second detection electrodes 21 and 22, which cut the sensing film and the detection electrodes together, the first and second detection electrodes are also exposed through the gap of the interlayer in the cross sections.

Referring to FIG. 5, which is a schematic structural diagram (front view) of a capacitive sensing film according to an embodiment of the present invention. The sensing film 20 includes a flexible substrate 25 and a waterproof protective layer 26. The detection electrode 22 is exposed in the gap of the interlayer between the flexible substrate 25 and the waterproof protective layer 26, and the exposed part is marked with 22c. From FIG. 5, the thickness relationship between the detection electrode, the waterproof protective layer and the flexible substrate in this embodiment can be roughly seen, wherein the exposed part 22c of the detection electrode 22 is thinner or much thinner than the waterproof protective layer 26, and the waterproof protective layer 26 is thinner or much thinner than the flexible substrate 25.

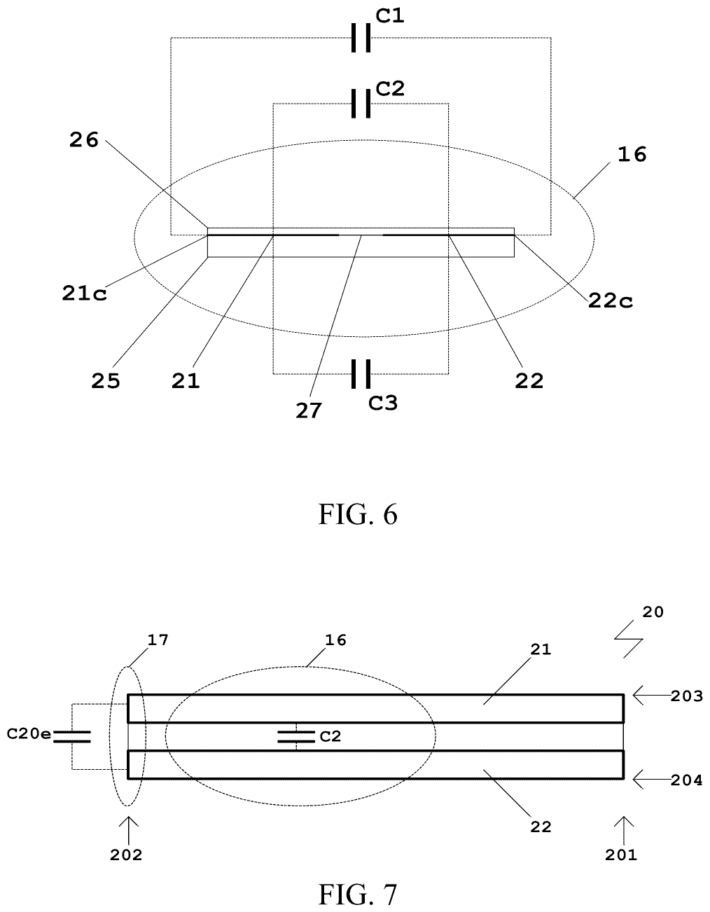

Referring to FIG. 6, this is a schematic diagram of the A-A′ cross sectional structure of the capacitive sensing film according to the embodiment of the present invention shown in FIG. 5 and an equivalent circuit diagram when fully wetted by a liquid containing electrolyte, it includes a flexible substrate 25, a waterproof protective layer 26, first and second detection electrodes 21, 22 and the portions 21c and 22c exposed on the longitudinal sections on the left and right sides of the sensing film. The figure also includes electrolyte-containing liquid 16, including salt-containing liquid, such as urine, loose stool, sweat, menstrual blood, etc., which may be called electrolyte solution or liquid for short. After the exposed parts 21c and 22c of the detection electrodes are wetted by the liquid, an electric double layer capacitance will be generated on the interface of the detection electrodes in contact with the liquid.

According to the electric double layer capacitance theory, when a liquid containing electrolyte is in contact with a solid electrode, an electric double layer capacitance will be generated on the contact interface between the solid electrode and the liquid (referred to as liquid-solid interface or interface). When a DC voltage is applied between the two electrodes, the negative ions in the liquid will accumulate on the positive electrode, the positive ions in the liquid will accumulate on the negative electrode, and the positive and negative ions in the liquid and the opposite ions on the solid electrode form an ionic dielectric, thereby generating an electric double layer capacitance.

It should be noted that the applied voltage should be smaller than the decomposition voltage of the liquid, otherwise the liquid will decompose, and a large current will be generated. For this embodiment, the electric double layer capacitance C1 can be detected between the first and second detection electrodes 21 and 22 (or between 21c and 22c), its capacitance is related to the surface area (including the outer surface area and the inner surface area) of the first and second detection electrodes in contact with the liquid, specifically, it is proportional to the length and thickness of the first and second detection electrodes 21 and 22 (i.e., the areas where the exposed parts 21c and 22c of the first and second detection electrodes are in contact with the liquid). Since the sensing film is designed in a strip shape, its longitudinal section is much longer than its cross section, therefore, in the embodiments of the present invention, the electric double layer capacitance on the cross section can be ignored.

At the same time, the liquid existing on the waterproof protective layer 26 will cause an electrolytic capacitance C2 to be generated between the first and second detection electrodes, which is proportional to the length and width (that is, the area) of the liquid on the waterproof protective layer corresponding to the first and second detection electrodes 21 and 22, proportional to the dielectric constant of the waterproof protective layer 26 and inversely proportional to the thickness of the waterproof protective layer 26. And the liquid existing on the flexible substrate 25 will cause the electrolytic capacitance C3 to be generated between the first and second detection electrodes, which is proportional to the length and width (i.e., the area) corresponding to the liquid on the flexible substrate corresponding to the first and second detection electrodes 21 and 22, proportional to the dielectric constant of the flexible substrate 25 and inversely proportional to the thickness of the flexible substrate 25.

To make the wetness detection have clear directionality, in the embodiment of the present invention, the thickness of the waterproof protective layer 26 is set to be smaller or much smaller than the thickness of the flexible substrate 25, so that the capacitance C2 is bigger or much bigger than C3, that is, the electrolytic capacitance generated between the first and second detection electrodes is mainly produced by liquid on the outer surface of the waterproof protective layer 26, and the electrolytic capacitance produced by the liquid on the outer surface of the flexible substrate 25 is small or even negligible, thus realizes the quantitative wetting detection function focusing on the direction of the waterproof protective layer. The quantification of the embodiment of the present invention refers to the specified amount, which can not only know the existence of a urine, but also estimate the amount of the urine according to the capacitance value. The amount of urine in this embodiment of the present invention is proportional to the electrolytic capacitance C2.

The electrolytic capacitance C2 of this embodiment of the present invention actually exists in the system shown in FIG. 7 of the prior art CN111077192A, but it does not reveal this fact, perhaps because the electrolytic capacitance is too small relative to the resulting electric double layer capacitance and is ignored. That is to say, the wetness detection between the first and second detection electrodes in the prior art is based on the electric double layer capacitance. In order to effectively suppress the electric double layer capacitance generated between the detection electrodes and increase the electrolytic capacitance generated between the detection electrodes, some key measures are adopted in the embodiment of the present invention, including:
1. Greatly reduce the thickness of the detection electrode. In the embodiment of the present invention, a nanoscale ultra-thin metal film is preferably used as the detection electrode, and its thickness is usually less than 1% of the traditional carbon electrode, so that the area/opportunity of the detection electrode exposed at the gap of the interlayer and contacting the electrolyte can be greatly reduced, thereby greatly reducing the generation of electric double layer capacitance on the contact interface.
2. Significantly reduce the surface area of the detection electrode material. Since the surface of the metal film electrode material is very smooth, its surface area is usually less than 1% of that of the carbon electrode material, therefore, the surface area of the metal film electrode in contact with the liquid can be greatly reduced, so that the generation of electric double layer capacitance on the contact interface can be greatly reduced. The traditional carbon electrode has a large surface area (including internal surface area), due to there are many micropores on its surface, when it is in contact with liquid, it will generate a large electric double layer capacitance (also called super capacitance).
3. Significantly reduce the thickness of the waterproof protective layer. The thickness of the waterproof protective layer in the embodiment of the present invention is usually only one tenth of the thickness of the commonly used flexible substrate, this can increase the ability of the liquid in the direction of the waterproof protective layer to generate electrolytic capacitors. In addition to improving the ratio of electrolytic capacitance to electric double layer capacitance, it also makes the detection of wetness have a clear directionality. That is to say, the electrolytic capacitance generated by the liquid on the outer surface of the waterproof protective layer is bigger or much bigger than the electrolytic capacitance generated by the liquid on the outer surface of the flexible substrate, so as to realize the wetness detection function focusing on the direction of the waterproof protective layer.

Through the above measures, the electric double layer capacitance (C1) of the embodiment of the present invention is reduced by about 4 orders of magnitude compared with the prior art solution, while the electrolytic capacitance (C2) is increased by about 1 order of magnitude, the addition of the two factors increases the ratio of the electrolytic capacitance output between the two detection electrodes to the electric double layer capacitance by about 5 orders of magnitude, so that the electrolytic capacitance C2 becomes the main detection parameter from the original negligible, and the electric double layer capacitance C1 is changed from the original main detection parameter to negligible, so the embodiment of the present invention realizes a leap from quantitative change to qualitative change, from the original electric double layer capacitive sensor to the electrolytic capacitive sensor.

In the embodiment of the present invention, the ratio of the thickness to the width of the detection electrode (thickness-to-width ratio) is a key parameter, the thinner the thickness and the wider the width of the electrode, the smaller the electric double layer capacitance it produces, and the bigger the electrolytic capacitance it produces. The ratio of electric double layer capacitance to electrolytic capacitance can be adjusted by adjusting the thickness-to-width ratio of the detection electrode. In the case of extremely small thickness-to-width ratio, the electric double layer capacitance generated on the cross section and longitudinal section of the sensing film will be much smaller than the electrolytic capacitance generated on the waterproof protective layer and can be ignored.

The metal film electrode of the embodiment of the present invention is not only much thinner than the carbon electrode, but also has a much lower resistance than the carbon electrode. Taking the detection electrode 10 mm wide and 1 meter long as an example, the resistance of the carbon electrode is about 300 kΩ, while the resistance of the vacuum evaporated aluminum film electrode is only about 300Ω, a reduction of about 1000 times. For the electrical conductivity of the printed electrode/film electrode, it can also be represented by the square resistance R=(ρ/d), where p is the resistivity of the electrode material and d is the thickness of the electrode. Since the resistivity of pure aluminum is about 2.83×10⁻⁸, the theoretical calculation value of the square resistance of 50 nanometer thick vacuum evaporated aluminum film electrode will be about 0.566 ohm, this square resistance is much smaller than that of conventional gravure printed conductive ink electrodes/carbon electrodes. In practical applications, the square resistance of metal film electrodes can usually be controlled between 0.1 and 50 ohms.

Conventional capacitance detection is to charge and discharge the capacitor through a specific resistor, then measure the voltage change between electrodes and use the formula c=it/v to calculate the capacitance value. In order to ensure the detection accuracy, the charge-discharge resistor is generally several times bigger than the maximum internal resistance of the detection loop (the sum of the resistances of the first and second detection electrodes). In the above example, the charge and discharge resistor of the carbon electrode is usually at the level of megohm when the capacitance detection is performed, such a high impedance can easily introduce electromagnetic interference, especially power frequency interference, which will cause the read data to jump and make the measurement results unreliable. In the embodiment of the present invention, the charge and discharge resistor only need several thousand ohms, under these circumstances, the electromagnetic interference/power frequency interference will be greatly reduced or effectively eliminated.

To sum up, the sensing film of the embodiment of the present invention uses the electrolytic capacitance as the main parameter for detection and output, and realizes the detection of wetness, it is essentially an electrolytic capacitive sensor, which is mainly based on the electrolytic capacitance C2 generated by the liquid on the waterproof protective layer 26, the electric double layer capacitance C1 generated at the gap of the interlayer of the sensing film will be effectively suppressed and weakened, even so small that it can be ignored. When the liquid fully wets the sensing film, the waterproof protective layer 26, the first detection electrode 21, the second detection electrode 22 together with the liquid containing electrolyte form an electrolytic capacitor with an electrolytic capacitance of C2, wherein the first and second detection electrodes 21 and 22 constitute the electrode plates of the capacitor, the waterproof protective layer 26 constitutes the dielectric of the capacitor, and the liquid constitutes the electrolyte (or liquid electrode) of the capacitor, the sensing film mainly expresses the quantitative wetness information by outputting the electrolytic capacitance C2.

For the convenience of description, C2 in the embodiment of the present invention is used to represent both the electrolytic capacitance generated between the first and second detection electrodes, and the corresponding electrolytic capacitor. Similarly, C1 is used to represent both the electric double layer capacitance generated between the first and second detection electrodes, and the electric double layer capacitor corresponding thereto. The same is true for other capacitance labels that have appeared or will appear.

The above embodiments are described by taking the sensing film including two detection electrodes as an example. In practical applications, it may also include more than two detection electrodes, for example, it may include 3 to 10 or more detection electrodes. When the sensing film is fully wetted by the liquid, a corresponding electrolytic capacitance will be generated between each detection electrode, as for the specifics are not repeated here.

Referring to FIG. 7, this is a schematic structural diagram (top view) of a capacitive sensing film in an embodiment of the present invention when a cross section is wetted by a liquid containing electrolyte. In the embodiment of the present invention, the first and second detection electrodes 21 and 22 are usually exposed through the incision/interlayer gap on the cross-sections of the head and tail ends, wherein the tail end (cross section 202) of the sensing film is usually arranged at the tail/back waist position of the disposable absorbent article, during the use of the disposable absorbent article, it is easily wetted by urine or back skin sweat 17 and will generate an electric double layer capacitance C20e between the electrodes, this electric double layer capacitance is undoubtedly a nonlinear mutation interference for the normal wetness detection based on the electrolytic capacitance C2, which will be superimposed on C2 and output together.

Because the structure of the embodiment of the present invention greatly suppresses the generation of electric double layer capacitance, the electric double layer capacitance C20e is much smaller than the electrolytic capacitance C2, in this way, the interference of electric double layer capacitance generated on the cross section of the sensing film can be effectively reduced or eliminated, and there will be no situation that the two detection electrodes are short-circuited by urine and the system cannot work normally. From another point of view, it can be considered that the sensing film of the embodiment of the present invention does not need to worry about liquid short circuit because of its superior ability to resist short circuit.

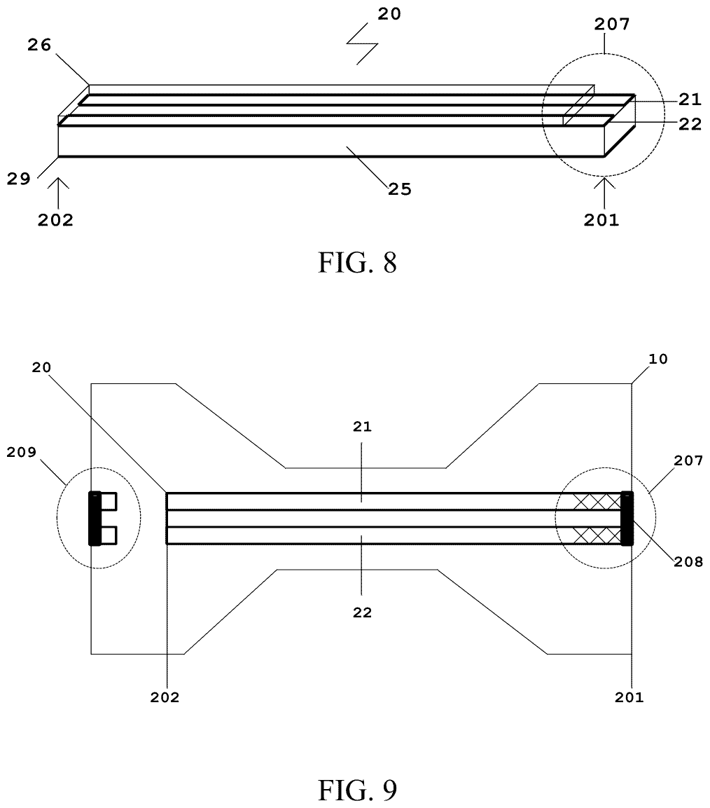

Referring to FIG. 8, this is a schematic structural diagram (a perspective view) when one end of a capacitive sensing film includes a bare area according to an embodiment of the present invention. The sensing film 20 of this embodiment includes a flexible substrate 25, first and second detection electrodes 21, 22, and a waterproof protective layer 26, and one end of the sensing film includes a bare area 207, since there is no waterproof protective layer in this area, the first and second detection electrodes will be directly exposed here, the user can set a detection device at this position and make electrical connection with the first and second detection electrodes 21 and 22, and then read the capacitance between the two electrodes through the detection device, thereby realizing the wetness detection function. In practical applications, the beginning (cross section 201) and the bare area 207 of the sensing film are usually set at the front end/front abdominal position of the disposable absorbent article, in this way, the detection device will not affect the sleep of the user of the disposable absorbent article, and the excrement will not wet the bare area at this position and affect the normal detection of the wetness.

In this embodiment, the outer surface of the flexible substrate 25 also includes another metal film layer (e.g., vacuum evaporated aluminum layer) 29, it can generate an initial capacitance C₀ proportional to the length of the sensing film between the first and second detection electrodes 21 and 22 of the sensing film, In addition, some soft composite layers or coating layers may be included on the outer surface of the flexible substrate 25 or on the vacuum evaporated aluminum layer 29 to make the sensing film 20 appear softer and more elastic as a whole, and the user will feel more comfortable when it is embedded in the disposable absorbent article.

Referring to FIG. 9, this is a schematic structural diagram (top view) of a capacitive sensing film including a positioning mark and disposed in a disposable absorbent article according to an embodiment of the present invention. The sensing film 20 of this embodiment of the present invention includes a bare area 207, where the first and second detection electrodes 21 and 22 are exposed because there is no waterproof protective layer in this area, as shown in the shaded portion in the figure. The bare area 207 is usually provided at the front end of the disposable absorbent article to avoid wetting by excreta, and the beginning (cross section 201) of the sensing film is flush with the front edge of the disposable absorbent article. The sensing film 20 of this embodiment is shorter than the disposable absorbent article 10, the advantage is that not only the sensing film can be saved, but also prevent the sweat of the user's back waist from interfering with the detection of urine by the sensing film.

In order to achieve precise positioning during the production of the disposable absorbent article, the sensing film of this embodiment further includes a color positioning mark 208, which can not only provide positioning service for the cutting of the sensing strip, but also provide positioning service for the cutting of the disposable absorbent article. When the disposable absorbent article is cut during the production process and becomes the final product, the sensing film is also cut simultaneously and divided into two sections, one long and one short, the long one is the main part, which includes the bare area 207 located at the front end of the disposable absorbent article and the sensing area located at the crotch of the disposable absorbent article; the short one is the residual part 209 generated after the sensing film is cut, which is located at the rear end of the disposable absorbent article, this part has no detection function, but a cutting error redundancy can be provided for the production of the disposable absorbent article to ensure that when the disposable absorbent article is cut with the sensing film, the beginning (cross-section 201) of the exposed area 207 of the sensing film can be flush with the front edge of the disposable absorbent article, and the beginning of the residual part 209 is flush with the rear edge of the disposable absorbent article. Such an arrangement is beneficial to clamp the detection device on the front edge of the disposable absorbent article, and to electrically connect with the first and second detection electrodes exposed in the exposed area of the sensing film, so as to realize corresponding wetness detection function.

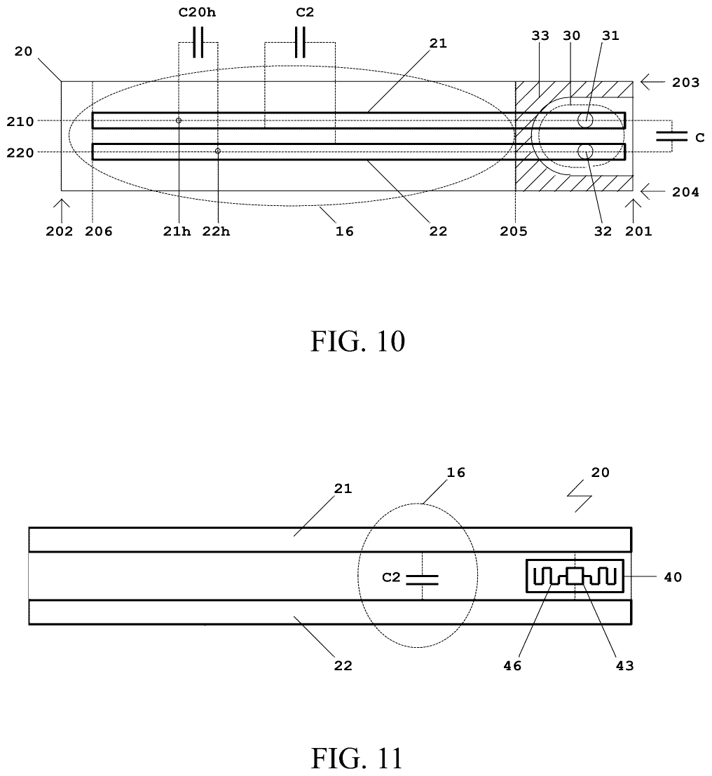

Referring to FIG. 10, this is a schematic structural diagram (top view) when the main body of the detection electrode of a capacitive sensing film is completely covered and sealed according to an embodiment of the present invention. Different from the situation in which the detection electrodes in the other aforementioned embodiments can be exposed through the gap of the interlayer in the longitudinal section of the sensing film, the main body of the first and second detection electrodes 21 and 22 (the section from 202 to 205) in this embodiment are completely covered and sealed by the flexible substrate and the waterproof protective layer, no detection electrodes are exposed at the gap of the interlayer at the outer edge of the sensing film 20.

In fact, it is not important whether the detection electrode of the embodiment of the present invention is exposed through the gap of the interlayer on the longitudinal section, and it basically has no effect on the detection of wetness, because the embodiment of the present invention does not rely on the detection electrode being exposed to contact with the liquid to generate an electric double layer capacitance to realize the detection of wetness, but an electrolytic capacitance is generated through the waterproof protective layer to realize the detection of the wetness. There is also no detection electrode near the end of the sensing film (cross section 202) in this embodiment, which has some advantages, because the end of the sensing film is usually set at the back waist of the disposable absorbent article, when the user is lying flat, the back waist is prone to sweating, there is no detection electrode in this place, which can effectively prevent the user's sweat from affecting the wetness detection of the disposable absorbent article.

Except for the sealed main body, this embodiment also includes a bare area near the beginning of the sensing film (cross section 201), a detection device 30 can be set here and make its contacts 31, 32 contact with the first and second detection electrodes 21, 22 to implement wetness detection. In order to fix and protect the detection device, this embodiment also includes a waterproof overlay 33, which can be formed of materials such as non-woven fabric, plastic film or waterproof paper, etc., it covers the bare area of the sensing film, Its three sides are bonded to other material below (the shaded part in the figure), and the middle part includes a hollow part, which can form a pocket for accommodating the detection device 30. The hollow part in the figure is U-shaped, which can be called a U-shaped pocket, the detection device 30 can be inserted into the U-shaped pocket from the U-shaped opening.

Due to the presence of the waterproof cover 33, this area of the sensing film from 201 to 205 cannot be used to detect wetness. In addition, this area of the sensing film from 202 to 206 cannot be used to detect wetness either because there is no detection electrode. The above-mentioned area that cannot be used for detecting wetness is called the non-working area/non-sensing area of the sensing film. Correspondingly, this area of the sensing film from 205 to 206 has detection electrodes and is not blocked by any other objects, so it can be used to detect wetness. This area is called the working area/sensing area of the sensing film.

Since the first and second detection electrodes of the sensing film of this embodiment are not exposed through the gap of the interlayer at the edge of the sensing film and generate electric double layer capacitance, so under normal circumstances, when liquid 16 is present on the working area of the sensing film, only the electrolytic capacitance value C2 is generated between the first and second detection electrodes 21 and 22. However, since the sensing film is a very soft and relatively fragile film, the sensing film may be damaged during the production and use of disposable absorbent articles, for example, perforations, cracks, creases, scratches, etc. (it is called the damaged part hereinafter), in these cases, the detection electrode may be exposed at the damaged part.

For example, when the liquid containing electrolyte 16 covers the damaged part/broken hole 21h and 22h, electric double layer capacitance C20h will also be generated between 21h and 22h. At this time, the capacitance C output between the first and second detection electrodes 21 and 22 includes both the electrolytic capacitance component C2 and the electric double layer capacitance component C20h. Fortunately, since the sensing film of the embodiment of the present invention has strong electric double layer capacitance suppression ability and electrolytic capacitance enhancement ability, even if electric double layer capacitance C20h is generated, its value is also much smaller than C2, and will not affect the normal wetness detection, that is to say, the sensing film of the embodiment of the present invention has a strong ability to resist damage.

It is a basic requirement of the embodiment of the present invention that the electrolytic capacitance generated between the two detection electrodes is bigger or much bigger than the electric double layer capacitance. Since the ability of the detection electrode to generate electric double layer capacitance is related to the thickness of the detection electrode, and the electrolytic capacitance generated by the detection electrode is related to the width of the detection electrode, in order to meet the requirements of the above capacitance ratio, the ratio of the thickness to the width (thickness-to-width ratio) of the detection electrode in the embodiment of the present invention is usually very small, and the smaller the thickness-to-width ratio, the better the above-mentioned capacitance ratio requirement can be satisfied. The thickness of the first and second detection electrodes (metal film electrodes) in the embodiment of the present invention is usually between 5 and 250 nanometers, the width of the first and second detection electrodes is usually between 1 and 50 mm, the width of the sensing film is usually between 5 and 100 mm, that is to say, the thickness-to-width ratio of the detection electrode is about 1/4,000 to 1/10,000,000, preferably 1/10,000 to 1/1,000,000.

In order to check whether the sensing film meets the above requirements, the sensing film can be directly immersed in the liquid containing electrolyte for testing. For example, in this embodiment, the sensing area of the sensing film from 205 to 206 can be immersed in the liquid, the electrolytic capacitance generated between the first and second detection electrodes should be bigger or much bigger than the electric double layer capacitance this moment. As for the “much bigger” and “much smaller” in the embodiments of the present invention, it may be determined according to the accuracy requirements of practical applications, for example, 3 to 30 times or more may be selected as the criterion for being much bigger, and ⅓ to 1/30 or less may be selected as the criterion for being much smaller.

In order to check whether the sensing film meets the requirements in the case of damage, the sensing film 20 can also be longitudinally cut along the centerlines 210 and 220 of the first and second detection electrodes 21 and 22, so that the first and second detection electrodes can be exposed through the gap of the interlayer of the resulting longitudinal section, then soak the cut sensing film into the liquid containing electrolyte and measure the electric double layer capacitance and electrolytic capacitance between the first and second detection electrodes, if the electrolytic capacitance is bigger or much bigger than the electric double layer capacitance at this time, then the sensing film is considered to meet the basic requirements of the present invention for the sensing film.

FIG. 11 is a schematic structural diagram (top view) of a capacitive sensing film including an RFID tag according to an embodiment of the present invention. In addition to the first and second electrodes 21 and 22, the sensing film 20 of this embodiment also includes an RFID tag 40. The RFID tag 40 includes a RFID chip 43 and an RFID antenna 46, wherein the RFID chip 43 is electrically connected to the first and second detection electrodes 21 and 22 of the sensing film 20, when there is liquid containing electrolyte 16 on the waterproof protective layer of the sensing film, an electrolytic capacitance C2 will be generated between the two detection electrodes. When there is an RFID reader near the sensing film 20 and read the information of the RFID tag 40, the RFID tag 40 will be woken up, and will acquire the wetness information related to the electrolytic capacitance C2 from the first and second detection electrodes 21 and 22 through the RFID chip 43, and then send the relevant information to the outside through the RFID antenna 46 in the form of radio frequency, Thereby, a RFID based wireless wetness detection is realized.

Referring to FIG. 12, this is a schematic structure diagram of a capacitive sensing film including only one metal film electrode according to an embodiment of the present invention and an equivalent circuit diagram when it is fully wetted by a liquid containing electrolyte (cross sectional view). In this embodiment, the sensing film 23 may be a part cut out from a large roll of sensing film material, it may also be a portion cut out according to the size of the detection electrode from the sensing film 20 of the foregoing embodiments. The metal film electrode 21 in the cut-out sensing film 23 can be exposed through the section cutouts at the edge of the interlayer formed by the flexible substrate 251 and the waterproof protective layer 261.

The sensing film of this embodiment has a strip shaped design. When the sensing film 23 is immersed in the liquid 16, four capacitances will be generated between the metal film electrode and the liquid, the liquid 16 here can be regarded as an electrode plate of the corresponding capacitor and can also be called an electrolyte electrode or a liquid electrode. These four capacitances are respectively the electrolytic capacitance C21 generated between the detection electrode 21 and the liquid electrode 16 in the direction of the waterproof protective layer 261, the electrolytic capacitance C31 generated between the detection electrode 21 and the liquid electrode 16 in the direction of the flexible substrate 251, the electric double layer capacitance C11a generated between the detection electrode 21 and the liquid electrode 16 on the left longitudinal section of the sensing film, and the electric double layer capacitance C11b generated between the detection electrode 21 and the liquid electrode 16 on the right longitudinal section of the sensing film. In this embodiment, C11a and C11b are equal. This embodiment does not consider the electric double layer capacitance generated on the cross section of the sensing film. Since the sensing film is elongated, its cross section is much shorter than the longitudinal section, so the capacitance generated on the cross section can be ignored.

The capacitance generated between the metal film electrode and the liquid of this embodiment has a specific corresponding relationship with the capacitance generated between the first and second detection electrodes in other embodiments (e.g., the embodiment shown in FIG. 6). Assuming that the size of the first and second detection electrodes are the same, and the capacitance C3 is much smaller than the capacitance C2 and can be ignored, in this case, since only one side of the first and second detection electrodes of the sensing film 20 shown in FIG. 6 can be exposed through the gap of the interlayer, two electric double layer capacitances will be generated and connected in series, therefore, the electric double layer capacitance generated between the first and second detection electrodes will be equal to a quarter of the electric double layer capacitance generated between the sensing film 23 and the liquid in this embodiment, and the electrolytic capacitance generated between the first and second detection electrodes is equal to half of the electrolytic capacitance generated between the sensing film 23 and the liquid in this embodiment.

That is to say, if the electrolytic capacitance generated between the sensing film 23 and the liquid in this embodiment is bigger or much bigger than the electric double layer capacitance generated between the sensing film 23 and the liquid, then it can be guaranteed that the electrolytic capacitance generated between the first and second detection electrodes by the sensing film 20 of the foregoing embodiment is bigger or much bigger than the electric double layer capacitance generated between the first and second detection electrodes. In actual operation, two identical sensing films 23 can also be immersed in the liquid or cut the sensing film 23 along the center line of the length direction, divide the sensing film into two, and then soak the two cut sensing films into the liquid, and measure the electrolytic capacitance and electric double layer capacitance between the two detection electrodes corresponding to the two cut sensing films, if the electrolytic capacitance is bigger or much bigger than the electric double layer capacitance, the basic requirement for the capacitance ratio of the sensing film in the embodiment of the present invention is met.

It should be noted that when measuring the capacitance between the metal film electrode and the liquid, it is usually necessary to insert an additional detection electrode into the liquid to make the liquid a liquid electrode of the detection system, and the sensing film to be tested usually needs to include a part of the non-working area to connect the test instrument or detection device, therefore, immersing the sensing film in the liquid in the embodiment of the present invention generally refers to immersing the working area of the sensing film into the liquid, so as to prevent the liquid from short-circuiting the two connecting ends of the test instrument. From another point of view, the non-working area of the sensing film can also be considered as an extension of the sensing film, or it can be considered that the non-working area of the sensing film is not a part of the sensing film, but only a supporting part of the sensing film.

In addition, it should be noted that the working voltage output by the test instrument should be smaller than the decomposition voltage of the liquid, which must be paid attention to in the actual test operation. Additionally, some auxiliary equipment, tools or experimental methods may be required to distinguish the electric double layer capacitance from the electrolytic capacitance during the test process, and some preprocessing of the sensing film to be detected may also be required. Furthermore, the electrolytic capacitance and electric double layer capacitance between the metal film electrode and the liquid can be obtained and compared according to theoretical calculations. Since the above-mentioned specific operations are not the contents that need to be explained in the present invention, they will not be repeated here.

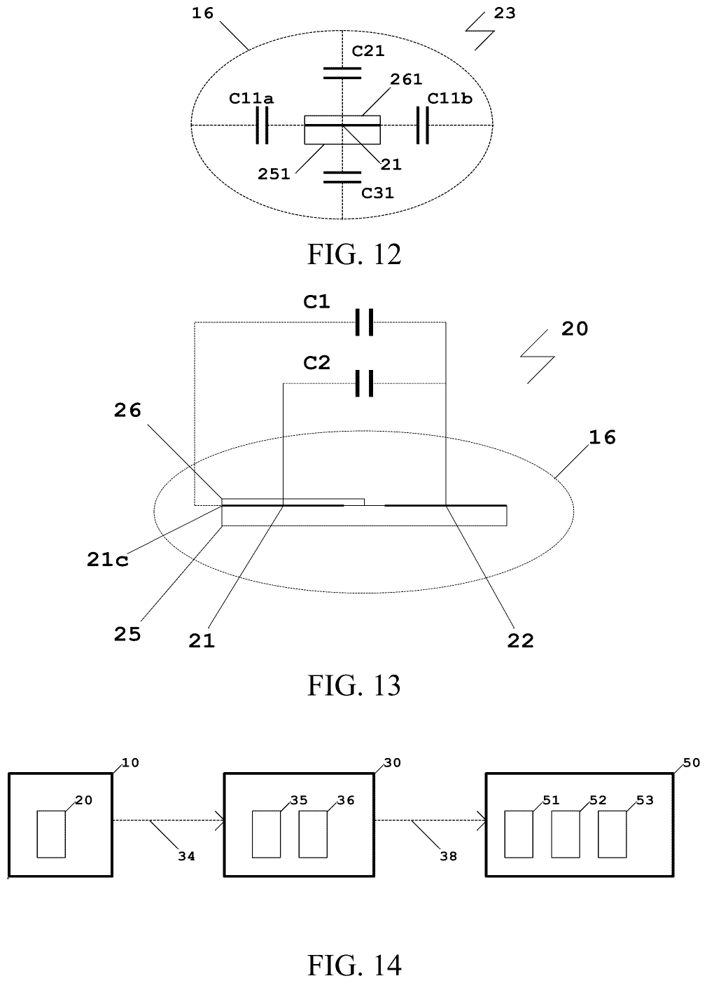

Referring to FIG. 13, this is a schematic structural diagram of a capacitive sensing film including a bare detection electrode and an equivalent circuit diagram (cross section view) when it is fully wetted by a liquid containing electrolyte according to an embodiment of the present invention. In this embodiment, the sensing film 20 includes a flexible substrate 25, a waterproof protective layer 26, and a first detection electrode 21 disposed in the interlayer formed by the flexible substrate and the waterproof protective layer, however, there is no waterproof protective layer on the second detection electrode 22, that is to say, the second detection electrode in this embodiment is bare, which can directly contact the liquid 16, the second detection electrode corresponds in this case to a connection or output of the liquid/liquid electrode 16. As with the other embodiments previously described, as long as the electrolytic capacitance C2 generated between the first and second detection electrodes is bigger than or much bigger than the electric double layer capacitance C1, the basic requirements of this embodiment of the present invention can be met.

Referring to FIG. 14, which is a functional block diagram of an intelligent wetness detection system according to an embodiment of the present invention. In the figure, the disposable absorbent article 10 and the sensing film 20 are included, and the combination of the disposable absorbent article 10 and the sensing film 20 constitutes a smart diaper. In this embodiment, a detection device 30 is further included, and the detection device 30 is electrically connected to the detection electrode of the sensing film 20 in the disposable absorbent article 10 through the electrical connection 34. Specifically, some probes or contacts can be set on the detection device 30 as contact electrodes, and the detection device can be clamped on the disposable absorbent article or inserted into the U-shaped pocket of the sensing film when in use, and the intelligent wetness monitoring function of the embodiment of the present invention can be realized by electrically connecting these probes or contacts with the first and second detection electrodes in the bare area on the sensing film 20.

The detection device 30 includes a capacitance detection unit 35, which can detect the capacitance between the detection electrodes of the sensing film in the smart diaper in real time and realize the wetness detection function based on electrolytic capacitance, and then transmit the wetness information, including alarm information, etc., in a wireless manner through the wireless transmitting device 36.

The relevant state information 38 is received by the wireless receiving device 50, and the wireless receiving device includes a wireless receiving unit 51, after receiving the relevant state information, the wireless receiving unit can display or indicate the status through the status display unit 52 or give an alarm prompt through the status alarm unit 53. In addition to dedicated wireless receiving and display devices (such as sound or light alarm devices), wireless receiving devices may also include a smart phone, a personal computer (PC) or a tablet computer (tablet), on which apps or software can be run, and the functions of wetness detection, data recording and query based on the capacitive sensing film and smart diaper according to the embodiment of the present invention are realized by the combination of software and hardware.

The above disclosures are only some preferred embodiments of the present invention, and the wetness detection of disposable absorbent articles is taken as an example to illustrate, and the scope of the rights of the present invention cannot be limited by this, therefore, the equivalent changes made according to the claims of the present invention and the expansion of the application scope still belong to the scope covered by the present invention.

1. A capacitive sensing film, comprising a metal film electrode, a flexible substrate and a waterproof protective layer, the metal film electrode comprise metal film layers formed on the flexible substrate by deposition, and the waterproof protective layer covers the metal film electrode and forms an interlayer together with the flexible substrate, adapted to protect the metal film electrode, the metal film electrodes has a smooth surface and a nanoscale thickness, adapted to reduce the surface area of the metal film electrode in contact with the liquid through the gap of the interlayer at the edge or damaged part of the capacitive sensing film, thereby inhibiting the generation of electric double layer capacitance at contact interface, when the capacitive sensing film is wetted by a liquid containing electrolyte, the electric double layer capacitance generated between the metal film electrode and the liquid is smaller than the electrolytic capacitance generated between the metal film electrode and the liquid, thus the interference of double layer capacitance is weakened and the wetness detection function based on electrolytic capacitance is realized.

2. The capacitive sensing film according to claim 1, wherein the metal film electrode comprises a metal film layer formed by physical vapor deposition, the metal film electrode have a strip shape and a very small thickness-to-width ratio, so as to increase its ability to generate electrolytic capacitance based on width and suppress its ability to generate electric double layer capacitance based on thickness, and make the capacitive sensing film have the ability to detect the degree of wetness by means of electrolytic capacitance; and
The capacitive sensing film comprises at least one cross section or longitudinal section, and the metal film electrode is exposed through a sandwich gap on the cross section or longitudinal section, when the capacitive sensing film is completely infiltrated by the liquid, the electrolytic capacitance generated between the metal film electrodes and the liquid is more than 3 times larger than the electric double layer capacitance generated between the metal film electrodes and the liquid on the sandwich gap of the cross section or longitudinal section; or
when the capacitive sensing film is cut along the middle line of the metal film electrode and the left and right two cut capacitive sensing films and the corresponding left and right metal film electrodes are obtained, the left and right metal film electrodes can be exposed through the sandwich gap on the incision, and when the left and right two incised capacitive sensing films are completely infiltrated by the liquid, the electrolytic capacitance generated between the left and right metal film electrodes is more than 3 times larger than the electric double layer capacitance generated between the left and right metal film electrodes.

3. The capacitive sensing film according to claim 2, wherein the metal film layer comprises a vacuum metal evaporation film layer, and the metal film electrode has a thickness to width ratio of less than 1/5000, which is used to suppress the electric double layer capacitance generated at the edge, incision or damage of the capacitive sensing film, so that the capacitive sensing film has the ability to resist short circuit and resist damage; and the metal film electrode has a square resistance of less than 50 ohms, which is used to suppress electromagnetic interference generated inside the metal film electrode, so that the capacitive sensing film has the ability to resist strong interference.

4. The capacitive sensing film according to claim 1, wherein the thickness of the waterproof protective layer is smaller than the thickness of the flexible substrate, adapted to improve the ability of the capacitive sensing film to generate electrolytic capacitance in the direction of the waterproof protective layer; when the capacitive sensing film is fully wetted by the liquid, the electrolytic capacitance generated by the liquid on the outer surface of the waterproof protective layer is bigger than the electrolytic capacitance generated by the liquid on the outer surface of the flexible substrate, thereby realizing the wetness detection function focusing on the direction of the waterproof protective layer.

5. The capacitive sensing film according to claim 4, wherein the flexible substrate comprises a plastic film, and the vacuum metal evaporation film layer comprises a vacuum evaporation aluminum film layer; the thickness of the metal film electrode is between 5 and 250 nm; the thickness of the waterproof protective layer is between 0.25 and 5 microns; the thickness of the flexible substrate is between 5 and 250 microns; the liquid containing electrolyte includes urine, loose stool, sweat, menstrual blood or saline; the waterproof protective layer comprises a polymer coating layer, a waterproof printing layer, or a plastic composite layer.

6. The capacitive sensing film according to claim 1, wherein the capacitive sensing film is in the shape of an elongated strip and comprises at least two of the metal film electrodes, forming a first detection electrode and a second detection electrode, of which at least one of the detection electrodes is located in an interlayer composed of the flexible substrate and the waterproof protective layer, when the capacitive sensing film is infiltrated by a liquid containing electrolyte, the electrolytic capacitance generated between the first and second detection electrodes is greater than the electric double layer capacitance generated between the first and second detection electrodes, and the capacitive sensing film is configured to achieve the wetness detection function by detecting the electrolytic capacitance between the first and second detection electrodes, and the wetness is proportional to the electrolytic capacitance between the first and second detection electrodes.

7. The capacitive sensing film according to claim 6, wherein the width of the capacitive sensing film is between 5 and 100 mm; the width of the first and second detection electrodes is between 1 and 50 mm; the length of the capacitive sensing film is between 5 and 120 cm; one end of the capacitive sensing film comprises an exposed area where the first and second detection electrodes are exposed for electrically connecting with a detection device and realizing wetness detection function.

8. The capacitive sensing film according to claim 7, further comprises a waterproof covering layer comprising a non-woven fabric, a plastic film, or a waterproof paper, which is covered over the exposed area and together with the exposed area forms a pocket accommodating the detection device.

9. The capacitive sensing film according to claim 6, further comprises an electronic tag, which comprises an electronic tag chip and an electronic tag antenna, the electronic tag chip is configured to be electrically connected with the first and second detection electrodes and to obtain wetness degree information through the electrolytic capacitance detection between the first and second detection electrodes; the electronic tag antenna is adapted to transmit the information outward through a radio frequency mode.

10. A smart diaper, comprising the capacitive sensing film according to claim 1, and a disposable absorbent article, which comprises a top layer, an absorbent layer and a leak-proof layer, and the capacitive sensing film is disposed in the disposable absorbent article and is configured to realize a quantitative wetness detection function by means of electrolytic capacitance detection.

11. The smart diaper of claim 10, wherein the capacitive sensing film comprises a color positioning mark, adapted to guide the production equipment to cut off the capacitive sensing film and the disposable absorbent article at a specific place, the capacitive sensing film is cut into two sections, one long section and one short section, the long section is the main part, and the short section is the residual part, the main part comprises a sensing area and a bare area, the sensing area is located at the crotch portion of the disposable absorbent article, the starting end of the bare area is flush with the front edge of the disposable absorbent article, and the starting end of the residual part is flush with the rear edge of the disposable absorbent article.

12. The smart diaper according to claim 10, wherein the disposable absorbent article includes a diaper, an insertion pad, a pull-up pant, a training pant, a sanitary napkin, a maternity napkin, or a urine pad; the absorption layer comprises polymer absorption material, the top layer comprises a hydrophilic non-woven fabric, and the leak-proof layer comprises a breathable or non-breathable polyethylene film.

13. A wetness detection system device, comprising a smart diaper as described in claim 10 and a detection device, wherein the detection device is electrically connected with the metal film electrode in the smart diaper for realizing the quantitative wetness detection function based on the smart diaper by means of electrolytic capacitance detection.

14. The wetness detection system device according to claim 13, wherein the detection device comprises a wireless transmitting device and a wireless receiving device for transmitting and receiving the wetness information of the disposable absorbent article and adapted to realize a wetness indication or a wetness alarm function.

15. The wetness detection system device according to claim 14, wherein the wireless receiving device comprises a dedicated sound or light alarm device, a smartphone, a PC, or a tablet.
